(19) United States
(12) Patent Application Publication
(10) Pub. No.: US 2020/0380957 A1
(43) Pub. Date: Dec. 3, 2020

(54) Systems and Methods for Machine Learning of Voice Attributes

(71) Applicant: Insurance Services Office, Inc., Jersey City, NJ (US)

(72) Inventors: Erik Edwards, Oakland, CA (US); Shane De Zilwa, Oakland, CA (US); Nicholas Irwin, Hallandale Beach, FL (US); Amir Poorjam, Copenhagen (DK); Flavio Avila, Oakland, CA (US); Keith L. Lew, Larchmont, NY (US); Christopher Sirota, Brooklyn, NY (US)

(73) Assignee: Insurance Services Office, Inc., Jersey City, NJ (US)

(21) Appl. No.: 16/889,307

(22) Filed: Jun. 1, 2020

Related U.S. Application Data
(60) Provisional application No. 62/854,652, filed on May 30, 2019.
(60) Provisional application No. 62/989,485, filed on Mar. 13, 2020.
(60) Provisional application No. 63/018,892, filed on May 1, 2020.

(51) Int. Cl. G10L 15/16; G06N 3/08; G06N 3/04; G10L 25/24; G10L 25/66

(52) U.S. Cl. CPC G10L 15/16; G06N 3/08; G10L 25/66; G10L 25/24; G06N 3/04

(57) ABSTRACT

Systems and methods for machine learning of voice and other attributes are provided. The system receives input data, isolates predetermined sounds from isolated speech of a speaker of interest, summarizes the features to generate variables that describe the speaker, and generates a predictive model for detecting a desired feature of a person Also provided are systems and methods for detecting one or more attributes of a speaker based on analysis of audio samples or other types of digitally-stored information (e.g, videos, photos, etc.).

### RELATED APPLICATIONS

This application claims priority to U.S. Provisional Patent Application Ser. No. 62/854,652 filed on May 30, 2019, U.S. Provisional Patent Application Ser. No. 62/989,485 filed on Mar. 13, 2020, and U.S. Provisional Patent Application Ser. No. 63/018,892 filed on May 1, 2020, the entire disclosures of which are hereby expressly incorporated by reference.

### BACKGROUND

### Technical Field

The present disclosure relates generally to the field of machine learning technology. More specifically, the present disclosure relates to systems and methods for machine learning of voice attributes.

### Related Art

In the machine learning space, there is significant interest in developing computer-based machine learning systems which can identify various characteristics of a person's voice. Such systems are of particular interest in the insurance industry. As the life insurance industry moves toward increased use of accelerated underwriting, a major concern is premium leakage from smokers who do not self-identify as being smokers. For example, it is estimated that a 60-year-old male smoker will pay approximately $50,000 more in premiums for a 20-year term life policy than a non-smoker. Therefore, there is clear incentive for smokers to attempt to avoid self-identifying as smokers, and it is estimated that 50% of smokers do not correctly self-identify on life insurance applications. In response, carriers are looking for solutions to identify smokers in real-time, so that those identified as having a high likelihood of smoking can be routed through a more comprehensive underwriting process.

An extensive body of academic literature shows that smoking cigarettes leads to irritation of the vocal folds (e.g., vocal cords), which manifests itself in numerous changes to a person's voice, such as changes to the fundamental frequency, perturbation characteristics (e.g., shimmer and jitter), and tremor characteristics. These changes make it possible to identify whether an individual speaker is a smoker or not by analysis of their voice.

In addition to detecting voice attributes such as whether a speaker is a smoker, there is also tremendous value in being able to detect other attributes of the speaker by analysis of the speaker's voice, as well as analysis of other attributes such as video analysis, photo analysis, etc. For example, in the medical field, it would be highly beneficial to detect whether an individual is suffering from an illness based on evaluation of the individual's voice or other sounds emanating from the vocal tract, such as respiratory illnesses, neurological disorders, physiological disorders, and other impairment and conditions. Still further, it would be beneficial to detect the progression of the aforementioned conditions over time through periodic analysis of individuals' voices, and to undertake various actions when conditions of interest have been detected, such as physically locating the individual, providing health alerts to one or more individuals (e.g., targeted community-based alerts, larger broadcasted alerts, etc.), initiating medical care in response to detected conditions, etc. Moreover, it would be highly beneficial to be able to remotely conduct community surveillance and detection of illnesses and other conditions using commonly-available communications devices such as cellular telephones, smart speakers, computers, etc.

Therefore, there is a need for systems and methods for machine learning to learn voice and other attributes and to detect a wide variety of conditions and criteria relating to individuals and communities. These and other needs are addressed by the systems and methods of the present disclosure.

### SUMMARY

The present disclosure relates to systems and methods for machine learning of voice and other attributes. The system first receives input data, which can be human speech, such as one or more recordings of a person speaking (e.g., a monologue, a speech, etc.) and/or one or more conversations between two or more speakers (e.g., a recorded conversation, a telephone conversation, a Voice over Internet Protocol “VoIP” conversation, a group conversation, etc.). The system then isolates a speaker of interest by performing a speaker diarization which partitions an audio stream into homogeneous segments according to the speaker identity. Next, the system isolates predetermined sounds from the isolated speech of the speaker of interest, such as vowel sounds, to generate features. The features are mathematical variables describing the sound spectrum of the speaker's voice over small time intervals. The system then summarizes the features to generate variables that describe the speaker. Finally, the system generates a predictive model, which can be applied to vocal data to detect a desired feature of a person (e.g., whether or not the person is a smoker). For example, the system generates a modeling dataset comprising tags together with generated functionals, where the tags indicate a speaker's gender, age, smoker status (e.g., a smoker or a non-smoker), etc. The predictive model allows for modeling of a smoker status using smoker status tags as the target variables, and other tags (e.g., gender, age, etc.) as predictive variables.

Also provided are systems and methods for detecting one or more attributes of a speaker based on analysis of voice samples or other types of digitally-stored information (e.g, videos, photos, etc.). An audio sample of a person is obtained from one or more sources, such as pre-recorded samples (e.g., voice mail samples) or live audio samples recorded from the speaker. Such samples could be obtained using a wide variety of devices, such as a smart speaker, a smart phone, a personal computer system, a web browser, or other device capable of recording samples of a speaker's voice. The system processes the audio sample using a predictive voice model to detect whether a pre-determined attribute exists. If a pre-determined attribute exists, the system can indicate the attribute to the user (e.g., using the user's smart phone, smart speaker, personal computer, or other device), and optionally, one or more additional actions can be taken. For example, the system can identify the physical location of the user (e.g., using one or more geolocation techniques), perform cluster analysis to identify whether clusters of individuals exhibiting the same (or, similar) attribute exist and are located, broadcast one or more alerts, or transmit the detected attribute to one or more third-party computer systems (e.g., via secure transmission using encryption, or through some other secure means) for further processing. Optionally, the system can obtain further voice samples from the individual (e.g., periodically over time) in order to detect and track the onset of a medical condition, or progression of such condition.

### BRIEF DESCRIPTION OF THE DRAWINGS

The foregoing features of the invention will be apparent from the following Detailed Description, taken in connection with the accompanying drawings, in which:

FIG. 1 is a diagram illustrating the overall system of the present disclosure;

FIG. 2 is a flowchart illustrating overall process steps carried out by the system of the present disclosure;

FIG. 3 is a diagram showing the predictive voice model of the present disclosure applied to various disparate data;

FIG. 4 is a diagram illustrating sample hardware and software components capable of being used to implement the system of the present disclosure;

FIG. 5 is a flowchart illustrating additional processing capable of being carried out by the predictive voice model of the present disclosure;

FIG. 6 is a flowchart illustrating processing steps carried out by the system of the present disclosure for detecting one or more medical conditions by analysis of an individual's voice sample and undertaking one or more actions in response to a detected medical condition;

FIG. 7 is a flowchart illustrating processing steps carried out by the system for obtaining one or more voice samples from an individual;

FIG. 8 is a flowchart illustrating processing steps carried out by the system for performing various actions in response to one or more detected medical conditions; and

FIG. 9 is diagram illustrating various hardware components operable with the present invention.

### DETAILED DESCRIPTION

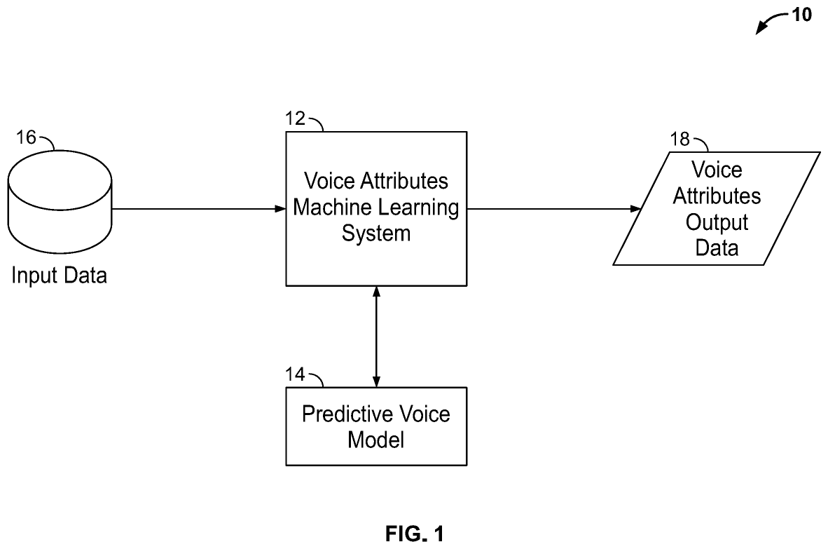

The present disclosure relates to systems and methods for machine learning of voice and other attributes, as described in detail below in connection with FIGS. 1-9. By the term “voice” as used herein, it is meant any sounds that can emanate from a person's vocal tract, such as the human voice, speech, singing, breathing, coughing, noises, timbre, intonation, cadence, speech patterns, or any other detectible audible signature emanating from the vocal tract.

FIG. 1 is a diagram illustrating the system of the present disclosure, indicated generally at 10. The system 10 includes a voice attributes machine learning system 12, which receives input data 16 and predictive voice model 14. The voice attributes machine learning system 12 and the predictive voice model 14 process the input data 16 to detect if a speaker has a predetermined characteristic (e.g., if the speaker is a smoker), and generate voice attribute output data 18. The voice attributes machine learning system 12 will be discussed in greater detail below. Importantly, the machine learning system 12 allows for the detection of various speaker characteristics with greater accuracy than existing systems. Additionally, the system 12 can detect voice components that are orthogonal to other types of information (such as the speaker's lifestyle, demographics, social medial, prescription information, credit information, allergies, medical conditions, medical issues, purchasing information, etc.).

The input data 16 can be human speech. For example, the input data 16 can be one or more recordings of a person speaking (e.g., a monologue, a speech, singing, breathing, other acoustic signatures emanating from the vocal tract, etc.), one or more conversations between two or more speakers (e.g., a recorded conversation, a telephone conversation, a Voice over Internet Protocol “VoIP” conversation, a group conversation, etc.). The input data 16 can be obtained from a dataset as well as from live (e.g., real-time) or recorded voice patterns of a speaker.

Additionally, the system 10 can be trained using a training dataset, such as a Mixer6 dataset from the Linguistic Data Consortium at the University of Pennsylvania. The Mixer6 dataset contains approximately 600 recordings of speakers in a two-way telephone conversation. Each conversation lasts approximately ten minutes. Each speaker in the Mixer6 dataset is tagged with their gender, age, and smoker status. Those skilled in the art would understand that the Mixer6 dataset is discussed by way of example, and that other datasets of one or more speakers/conversations can be used as the input data 14.

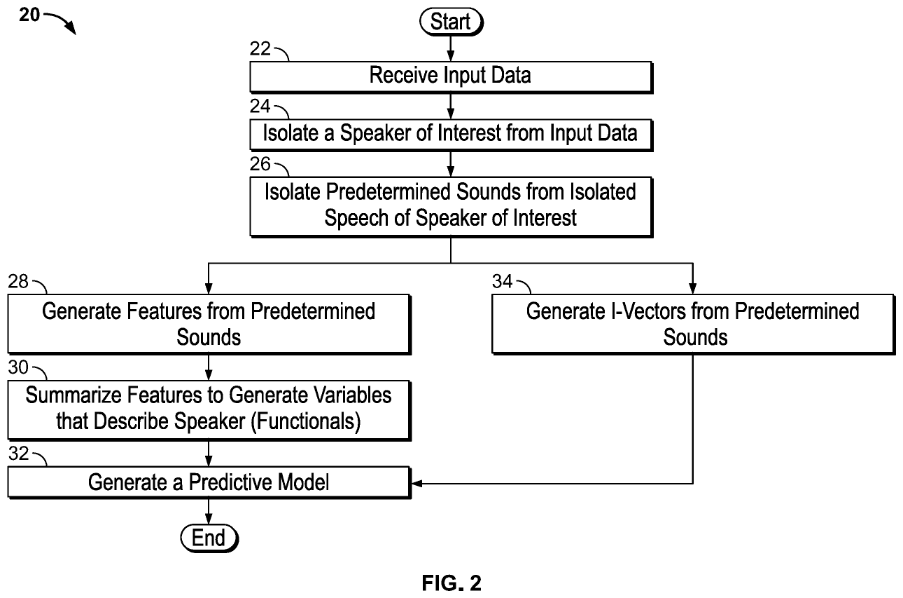

FIG. 2 is a flowchart illustrating the overall process steps being carried out by the system 10, indicated generally at method 20. In step 22, the system 10 receives input data 16. By way of example, the input data 16 could comprise telephone conversations between two speakers. In step 24, the system 10 isolates a speaker of interest (e.g., a single speaker). For example, the system 10 can perform a speaker diarisation (or diarization) process of partitioning an audio stream into homogeneous segments according to a speaker identity.

In step 26, the system 10 isolates predetermined sounds from the isolated speech of the speaker of interest. For example, the predetermined sounds can be vowel sounds. Vowel sounds disclose voice attributes better than most other sounds. This is demonstrated by a physician requesting a patient to make an “Aaaahhhh” sound (e.g., sustained phonation or clinical speech) when examining their throat. Voice attributes can comprise frequency, perturbation characteristics (e.g., shimmer and jitter), tremor characteristics, duration, timbre, or any other attributes or characteristics of a person's voice, whether within the range of human hearing, below such range (e.g., subsonic) or above such range (e.g., supersonic). The predetermined sounds can also include consonants, syllables, terms, guttural noises, etc.

In a first embodiment, the system 10 proceeds to step 28. In step 28, the system 10 generates features. The features are mathematical variables describing the sound spectrum of the speaker's voice over small time intervals. For example, the features can be mel-frequency cepstral coefficients (“MFCCs”). MFCCs are coefficients that make up a representation of the short-range power spectrum of a sound, based on a linear cosine transform of a log power spectrum on a nonlinear mel scale of frequency.

In step 30, the system 10 summarizes the features to generate variables that describe the speaker. For example, the system 10 aggregates the features so that each resultant summary variable (referred to as “functionals” hereafter) is at a speaker level. The functionals are, more specifically, features summarized over an entire record.

In step 32, the system 10 generates the predictive voice model 14. For example, the system 10 can generate a modeling dataset comprising tags together with generated functionals. The tags can indicate a speaker's gender, age, smoker status (e.g., a smoker or a non-smoker), etc. The predictive voice model 14 allows for predictive modeling of a smoker status, by using smoker status tags as the target variables, and other tags (e.g., gender, age, etc.) as predictive variables. The predictive voice model 14 can be a regression model, a support-vector machine (“SVM”) supervised learning model, a Random Forest model, a neural network, etc.

In a second embodiment, the system 10 proceeds to step 34. In step 34, the system 10 generates I-Vectors from predetermined sounds. I-vectors are the output of an unsupervised procedure based on a Universal Background Model (UBM). The UBM is a Gaussian Mixture Model (GMM) or other unsupervised model (e.g. deep belief network (DBN), etc.) that is trained on a very large amount of data (usually much more data than the labeled data set). The labeled data is used in the supervised analyses, but since it is only a subset of the total data available, it may not capture the full probability distribution expected from the raw feature vectors. The UBM recasts the raw feature vectors as posterior probabilities, and following a simple dimensionality reduction, the result is the I-vectors. This stage is also called “total variability modeling” since its purpose is to model the full spectrum of variability that might be encountered in the universe of data under consideration. Vectors of modest dimension (e.g., N-D) will not have their N-dimensional multivariate probability distribution adequately modeled by the smaller subset of labeled data, and as a result, the UBM utilizes the total data available, both labeled and unlabeled, to better fill in the N-D probability density function (PDF). This better prepares the system for the total variability of feature vectors that might be encountered during testing or actual use. The system 10 then proceeds to step 32 and generates a predictive model. Specifically, the system 10 generates the predictive voice model 14 using the I-Vectors.

The predictive voice model 14 can be implemented to detect a speaker's smoker status, as well as other speaker characteristics (e.g., age, gender, etc.) In an example, the predictive voice model 14 can be implemented in a telephonic system, a device that records audio, a mobile app, etc., and can process conversations between two speakers, (e.g., an insurance agent and a interviewee) to detect the interviewee's smoker status. Additionally, the systems and methods disclosed in the present disclosure can be adapted to detect further features of a speaker, such as age, deception, depression, stress, general pathology, mental and physical health, diseases (such as Parkinson's), and other features.

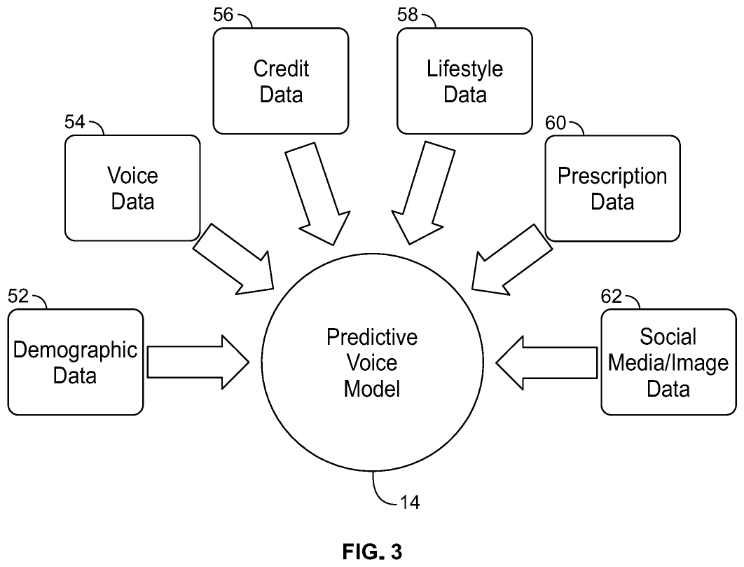

FIG. 3 is a diagram illustrating the predictive voice model 14 applied to various disparate data. For example, the predictive voice model 14 can process demographic data 52, voice data 54, credit data 56, lifestyle data 58, prescription data 60, social media/image data 62, or other types of data. The various disparate data can be processed by the system and methods of the present disclosure to determine features (e.g., smoker, age, etc.) of the speaker.

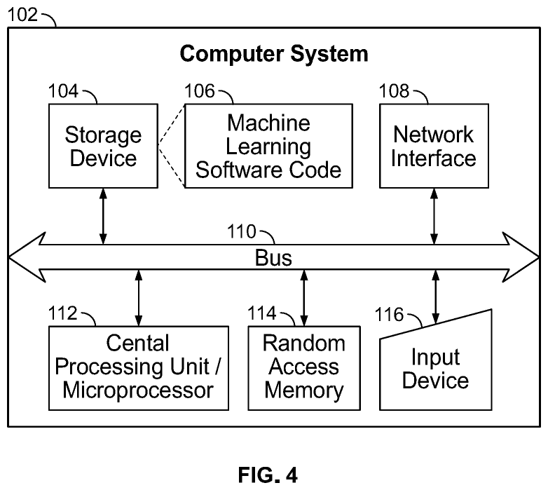

FIG. 4 is a diagram showing a hardware and software components of a computer system 102 on which the system of the present disclosure can be implemented. The computer system 102 can include a storage device 104, machine learning software code 106, a network interface 108, a communications bus 110, a central processing unit (CPU) (microprocessor) 112, a random access memory (RAM) 114, and one or more input devices 116, such as a keyboard, mouse, etc. The computer system 102 could also include a display (e.g., liquid crystal display (LCD), cathode ray tube (CRT), etc.). The storage device 104 could comprise any suitable, computer-readable storage medium such as disk, non-volatile memory (e.g., read-only memory (ROM), eraseable programmable ROM (EPROM), electrically-eraseable programmable ROM (EEPROM), flash memory, field-programmable gate array (FPGA), etc.). The computer system 102 could be a networked computer system, a personal computer, a server, a smart phone, tablet computer etc. It is noted that the computer system 102 need not be a networked server, and indeed, could be a stand-alone computer system.

The functionality provided by the present disclosure could be provided by the software code 106, which could be embodied as computer-readable program code stored on the storage device 104 and executed by the CPU 112 using any suitable, high or low level computing language, such as Python, Java, C, C++, C#, R, .NET, MATLAB, as well as tools such as Kaldi and OpenSMILE. The network interface 108 could include an Ethernet network interface device, a wireless network interface device, or any other suitable device which permits the server 102 to communicate via the network. The CPU 112 could include any suitable single-core or multiple-core microprocessor of any suitable architecture that is capable of implementing and running the machine learning software code 106 (e.g., Intel processor). The random access memory 114 could include any suitable, high-speed, random access memory typical of most modern computers, such as dynamic RAM (DRAM), etc.

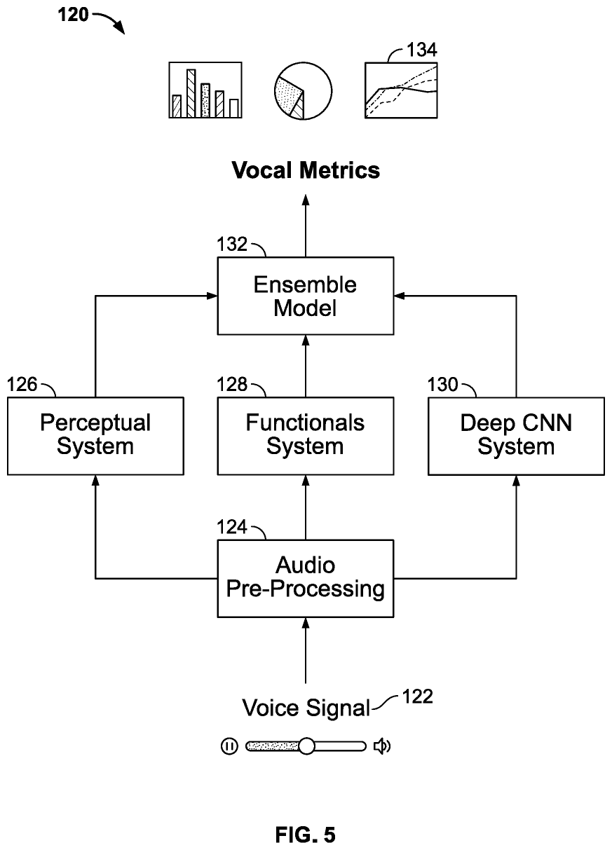

FIG. 5 is a flowchart illustrating additional processing capable of being carried out by the predictive voice model of the present disclosure, indicated generally at 120. As can be seen, an input voice signal 122 is obtained and processed by the system of the present disclosure. As will be discussed in greater detail below, the voice signal 122 could be obtained from a wide variety of sources, such as pre-recorded voice samples (e.g., from a person's voice mail box, from a recording specifically obtained from the person, or from some other source, including social media postings, videos, etc.). Next, in step 124, an audio pre-processing step is performed on the voice signal 122. This step can involve digital signal processing (DSP) of the signal 122, audio segmentation, and speaker diarization. It is noted that additional “quality control” pre-processing steps could be carried out, such as detecting outliers which do not include relevant information for voice analysis (e.g., the sound of a dog barking), detection and degredation in the voice signal, and signal enhancement. Such quality control steps can ensure that the received signal contains relevant information for processing, and that it has the acceptable quality. Speaker diarization determines “who spoke when,” such that the system labels each point in time according to the speaker identity. Of course, speaker diarization may not be required where the voice signal 122 contains only a single speaker.

Next, three parallel subsystems (an “ensemble”) are applied to the pre-processed audio signal, including a perceptual system 126, a functionals system 128, and a deep convolutional neural network (CNN) subsystem 130. The perceptual system 126 applies human auditory perception and classical statistical methods for robust prediction. The functionals system 128 generates a large number of derived functions (various nonlinear feature transformations), and machine learning methods of feature selection and recombination are used to isolate the most predictive subsets. The deep CNN subsystem 130 applies one or more CNNs (which are often utilized in computer vision) to the audio signal. Next, in step 132, an ensemble model is applied to the outputs of the subsystems 126, 128, and 130 to generate vocal metrics 134. The ensemble model takes the posterior probabilities of the subsystems 126, 128, and 130 and their associated confidence scores and combines them to generate a final prediction. It is noted that the process steps discussed in FIG. 5 could also account for auxiliary information known about the subject (the speaker), in addition to voice-derived features.

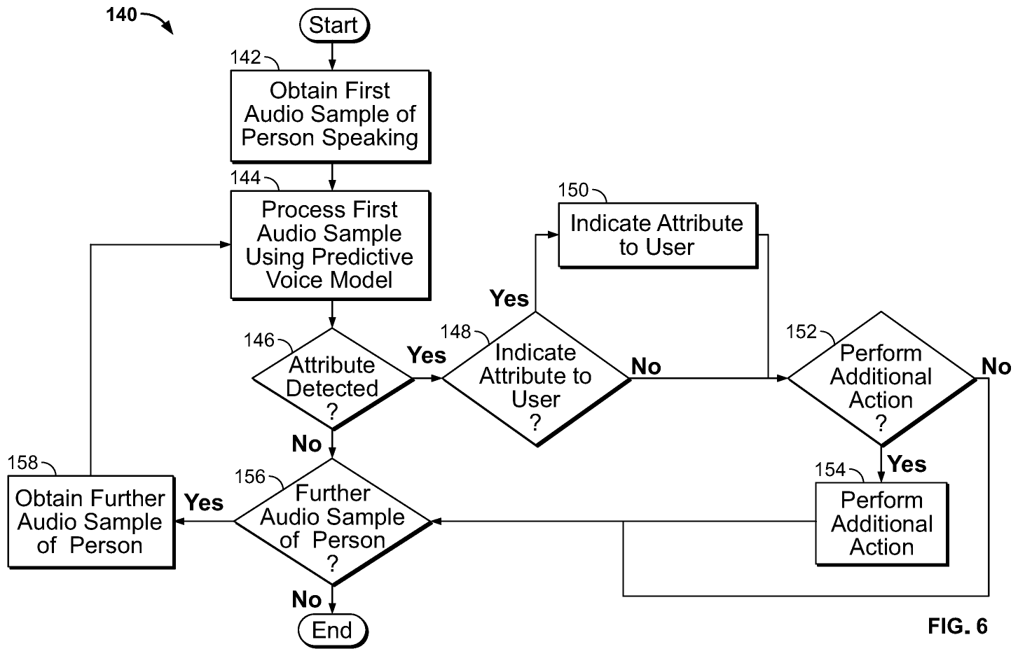

The processing steps discussed herein could be utilized as a framework for many voice analytics questions. Also, the processing steps could be applied to detect a wide variety of characteristics beyond smoker verification, such as age (prebyphonia), gender, general vocal pathology, regional accent, body size, attractiveness, sexuality, social status, personality, emotion, deception, sleepiness, hydration, stress, depression, Sjögren's syndrome, arthritis, dementia, Parkinson's disease, schizophrenia, reflux, alcohol intoxication, epidemiology, cannabis intoxication, blood oxygen levels, and a wide variety of medical conditions as will discussed herein in connection with FIG. 6.

FIG. 6 is a flowchart illustrating processing steps, indicated generally at 140, carried out by the system of the present disclosure for detecting one or more pre-determined attributes by analysis of an individual's voice sample and undertaking one or more actions in response to a detected attributes. The processing steps described herein can be applied to detect a wide variety of attributes based on vocal analysis, including, but not limited to, medical conditions such as respiratory symptoms, ailments, and illnesses (e.g., common colds, influenza, COVID-19, pneumonia, or other respiratory illnesses), neurological illnesses/disorders (e.g., Alzheimer's disease, Parkinson's disease, dementia, schizophrenia, etc.), moods, ages, physiological characteristics, or other any other attribute that manifests itself in perceptible changes to a person's voice.

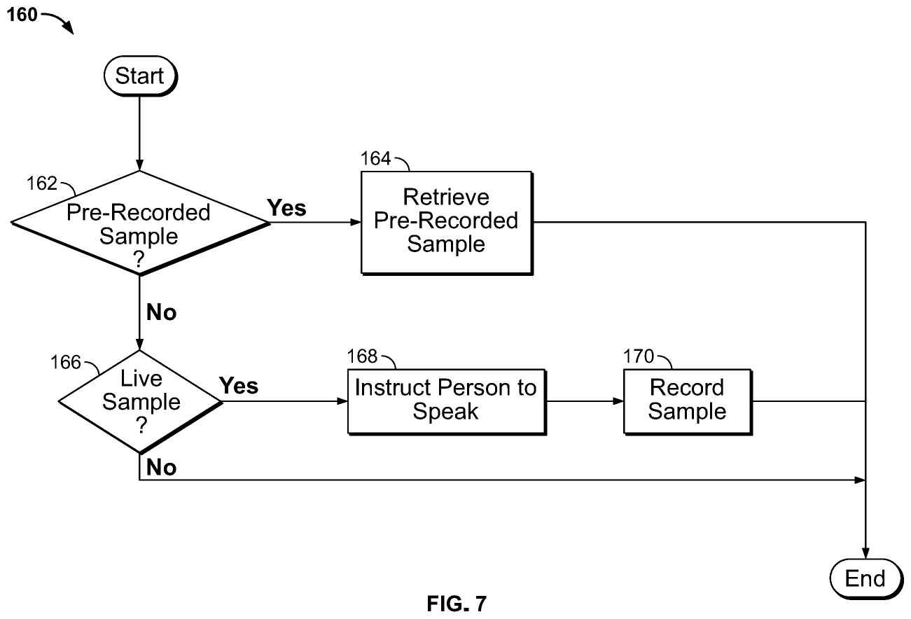

Beginning in step 142, the system obtains a first audio sample of a person speaking. As will be discussed in FIG. 7, there are a wide variety of ways in which the audio sample can be obtained. Next, in step 144, the system processes the first audio sample using a predictive voice model, such as the voice models disclosed herein. This step could also involve saving the audio sample in a database of audio samples for future usage and/or training purposes, if desired. In step 146, based on the outputs of the predictive voice model, the system determines whether a predetermined attribute (such as, but not limited to, a medical condition) is detected. Optionally, the system could also determine the severity of such attribute. If a positive determination is made, step 148 occurs, wherein the system determines whether the detected attribute should be indicated to the user. If a positive determination is made, step 150 occurs, wherein the system indicates the detected medical condition to the user. The indication could be made in various ways, such as by displaying an indication of the condition on a user's smart phone or on a computer screen, audibly conveying the detected condition to the user (e.g., by a voice prompt played to the user on his or her smart phone, over a smart speaker, using the speakers of a computer system, etc.), transmitting a message containing an indication of the detected condition to the user (e.g., an e-mail message, a text message, etc.), or through some other mode of communication. Advantageously, such attributes can be processed by the system in order to obtain additional relevant information about the individual, or to triage medical care for the individual based on one or more criteria, if needed.

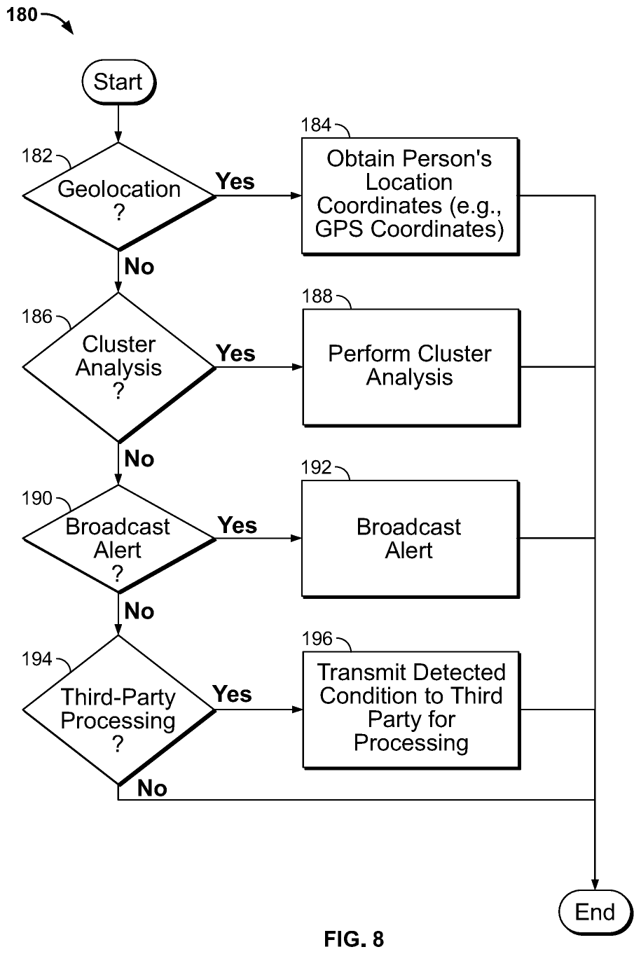

In step 152, a determination is made as to whether an additional action responsive to the detected attribute should occur. If so, step 154 occurs, wherein the system performs one or more additional actions. Examples of such actions are described in greater detail below in connection with FIG. 8. In step 156, a determination is made as to whether a further audio sample of the person should be obtained. If so, step 158 occurs, wherein the system obtains a further audio sample of the person, and the processing steps discussed above are repeated. Advantageously, by processing further audio samples of the person (e.g., by periodically asking the person to record their voice, or by periodically obtaining updated stored audio samples from a source), the system can detect both the onset, as well as the progression, of a medical condition being experienced by the user. For example, if the system detects (by processing of the initial audio sample) that the person has a viral disease such as COVID-19 (or that the person currently has attributes that are associated with such disease), processing of subsequent audio samples of the person (e.g., an audio sample of the person one or more days later) can provide an indication of whether the person is improving or whether more urgent medical care is required.

FIG. 7 is a flowchart illustrating data accquisition steps, indicated generally at 160, carried out by the system for obtaining one or more voice samples from an individual. As noted above in connection with step 142 of FIG. 6, there are a wide variety of ways in which the system can obtain audio samples of a person's voice. In step 162, the system determines whether the sample of the person's voice should be obtained from a pre-recorded sample. If so, step 164 occurs, wherein the system retrieves a pre-recorded sample of the person's voice. This could be obtained, for example, from a recording of the person's voice mail greeting, from a recorded audio sample or video clip posted on a social media platform or other service, or some other previously-recorded sample of the person's voice (e.g., one or more audio samples stored in a database). Otherwise, step 166 occurs, wherein a determination is made as to whether to obtain a live sample of the person's voice. If so, step 168 occurs, wherein the person is instructed to speak, and then in step 170, the system records a sample of the person's voice. For example, the system could prompt the person to speak a short or longer phrase (e.g., the Pledge of Allegience) using an audible or visual prompt (e.g., displayed on a screen of the person's smart phone, or audible prompting via voice synthesis or pre-recorded prompt), the person could then speak the phrase (e.g., into the microphone of the person's smart phone, etc.), and the system could record the phrase. The processing steps discussed in connection with FIG. 7 could also be used to obtain future samples of the person speaking, such as in connection with step 158 of FIG. 6, to allow for future monitoring and detection of medical conditions (or the progression thereof) being experienced by the person.

FIG. 8 is a flowchart illustrating action handling steps, indicated generally at 180, carried out by the system for performing various actions in response to one or more detected attributes. As noted above in connection with step 154 of FIG. 6, a wide variety of actions could be taken. For example, beginning in step 182, a determination could be made as to whether to determine physical location (geolocation) of the person in response to detection of an attribute, such as a medical condition. If so, step 184 occurs, wherein the system obtains the location of the person (e.g., GPS coordinates determined by polling a GPS receiver of the person's smart phone, the person's mailing or home address as stored in a database, radio frequency (RF) triangulation of cellular telephone signals to determine the user's location, etc.).

In step 186, a determination could be made as to whether to perform cluster analysis in response to detection of an attribute, such as, but not limited to, a medical condition. If so, step 188 occurs, wherein the system performs cluster analysis. For example, if the system determines that the person is suffering from a highly-communicable illness such as influenza or COVID-19, the system could consult a database of individuals who have previously been identified as having the same, or similar, symptoms as the person, determine whether such individuals are geographically proximate to the person, and then determine or one more geographic regions or “clusters” as having a high density of instances of the illness. Such information could be highly-valuable to healthcare professionals, government officials, law enforcement officials, and others in establishing effective quarantines or undertaking other measures in order to isolate such clusters of illness and prevent further spreading of the illness.

A determination could be made in step 190 whether to broadcast an alert in response to a detected attribute. If so, step 192 occurs, wherein an alert is broadcast. Such an alert could be targeted to one or more individuals, to small groups of individuals, to large groups of individuals, to one or more government or health agencies, or to other entities. For example, if the system determines that the individual has a highly-communicable illness, a message could be broadcast to other individuals who are geographically proximate to the individual or related to the individual, indicating that measures should proactively be taken to prevent further spreading of the illness. Such an alert could be issued by e-mail, text message, audibly, visually, or through any other means.

A determination could be made in step 194 whether further processing of the detected attribute should be transmitted to a third party for further processing. Such transmission could ber performed securely, using encryption or other means. If so, step 196 occurs, wherein the detected condition is transmitted to the third party for further processing. For example, if the system detects that an individual has a cold (or that the individual is exhibiting symptoms indicative of a cold), an indication of the detected condition could be sent to a healthcare provider so that an appointment for a medical examination is automatically scheduled. Also, the detected condition transmitted to a government or industry research entity for further study of the detected condition, if desired. Of course, other third-party processing of the detected condition could be performed, if desired.

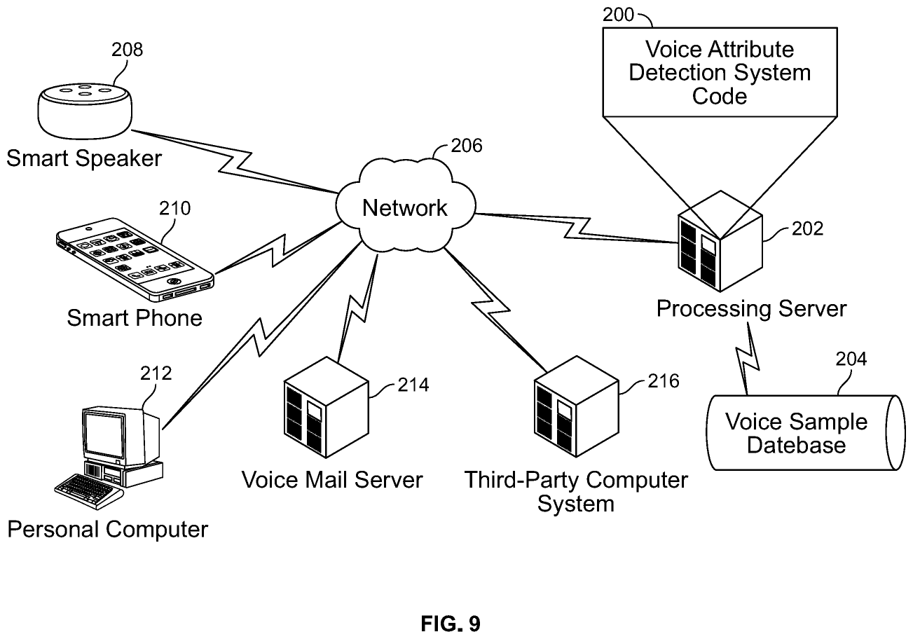

FIG. 9 is diagram illustrating various hardware components operable with the present invention. The system could be embodied as voice attribute detection software code 200 executed by a processing server 202. Of course, it is noted that the system could utilize one or more portable devices (such as smart phones, computers, etc.) as the processing devices for the system. For example, it is possible that a user can download a software application capable of carrying out the features of the present disclosure to his or her smart phone, which can perform all of the processes disclosed herein, including, but not limited to, detecting a speaker attribute and taking appropriate action, without requiring the use of a server. The server 202 could access a voice sample database 204, which could store pre-recorded voice samples. The server 202 could communicate (securely, if desired, using encryption or other secure communication method) with a wide variety of devices over a network 206 (including the Internet), such as a smart speaker 208, a smart phone 210, a personal computer or tablet computer 212, a voice mail server 214 (for obtaining samples of a person's voice from a voice mail greeting), or one or more third-party computer systems 216 (including, but not limited to, a government computer system, a health care provider computer system, an insurance provider's computer system, a law enforcement computer system, or other computer system). In one example, a person could be prompted to speak a phrase by the smart speaker 208, the smart phone 210, or the personal computer 212. The phrase could be recorded by either device and transmitted to the processing server 202, or streamed in real time to the processing server 202. The server 202 could store the phrase in the voice sample database 204, and process the phrase using the system code 200 to determine any of the attributes discussed herein of the speaker (e.g., if the speaker is a smoker, if the speaker is suffering an illness, characteristics of the speaker, etc.). If an attribute is detected by the server 202, the system could undertake any of the actions discussed herein (e.g., any of the actions discussed above in connection with FIGS. 6-8). Still further, it is noted that the embodiments of the system as described in connection with FIGS. 6-9 could also be applied to the smoker identification features discussed in connection with FIGS. 1-5.

It is noted that the voice samples discussed herein could be time stamped by the system so that the system can account for the aging of a person that may occur between recordings. Still further, the voice samples could be obtained using a customized software application (“app”) executing on a computer system, such as a smart phone, tablet computer, etc. Such an app could prompt the user visually as to what to say, and when to begin speaking. Additionally, the system could detect abnormalities in physiology (e.g., lung changes) that are conventionally detected by imaging modalities (such as computed tomography (CT) imaging) by analysis of voice samples. Moreover, by performing analysis for voice samples, the system can discern between degrees of illnesses, such as mild cases of illness and full (critical) cases. Further, the system could operate on a simpler basis, such that it determines from analysis of voice samples whether a person is sick or not. Even further, processing of voice samples by the system could ascertain whether the person is currently suffering from allergies.

An additional advantage of the systems and methods of the present disclosure is that it allows healthcare professionals to determine whether in-person treatment or testing is unavailable, unsafe, or impractical. Additionally, it is envisioned that the information obtained by the system of the present disclosure could be coupled with other types of data, such as biometric data, medical records, weather/climate data, imagery, calendar information, self-reported information (e.g., health, wellness, or mood information) or other types of data, so as to enhance monitoring and treatment, detection of infection paths and patterns, triaging of resources, etc. Even further, the system could be utilized by an employer or insurance provider to verify that an individual who claims to be ill is actually suffering an illness. Further, the system could be used by an employer to determine whether to hire an individual who has been identified as suffering an illness, and the system could also be used to track, detect, and/or control entry of sick individuals into businesses or venues (e.g., entry into a store, amusement parks, office buildings (including staff and employees of such buildings), other venues, etc.) as well as to ensure compliance with local health codes by businesses. Still further, the system could be used to aid in screening of individuals, such as airport screenings, etc., and to assist with medical community surveillance and diagnosis. Also, it is envisioned that the system could operate in conjunction with weather data and imagery data to ascertain regions where allergies or other illnesses are likely to occur, and to monitor individual health in such regions. In this regard, the system could obtain seasonal allergy level data, aerial imagery of trees or other foliage, information about grass, etc., in order to predict allergies. Further, the system could process aerial or ground-based imagery phenotyping data as well. Such information, in conjunction with detection of vocal attributes performed by the system, could be utilized to ascertain whether an individual is suffering from one or more allergies, or to isolate specific allergies by tying them to particular active allergens. Also, the system could process such information to control for allergies (e.g., to determine that the detected attribute is something other than an allergic reaction) or to diagnose allergies.

As noted above, the system can process recordings of various acoustic information emanating from a person's vocal tract, such as speech, signing, breath sounds, etc. With regard to coughing, the system could also process one or more audio samples of the person coughing, and analyze such samples using the predictive models discussed herein in order to determine the onset of, presence of, or progression of, one or more illnesses or medical conditions.

The systems and methods described herein could be integrated with, or operate with, various other systems. For example, the system could operate in conjunction with existing social media applications such as FACEBOOK to perform contact tracing or cluster analysis (e.g., if the system determines that an individual has an illness, it could consult a social media application to identify individuals who are in contact with the individual and use the social media application to issue alerts, etc.). Also, the system could integrate with existing e-mail application such as OUTLOOK in order to obtain contact information, transmit information and alerts, etc. Still further, the system of the present disclosure could obtain information about travel manifests for airplanes, ports of entry, security check-in times, public transportation usage information, or other transportation-related information, in order to tailor alerts or warnings relating to one or more detected attributes (e.g., in response to one or more medical conditions detected by the system).

It is further envisioned that the systems and methods of the present disclosure can be utilized in connection with authentication applications. For example, the various voice attributes detected by the systems and methods of the present disclosure could be used to authenticate the identity of a person or groups of people, and to regulate access to public spaces, government agencies, travel services, or other resources. Further, usage of the systems and methods of the present disclosure could be required as a condition to allow an individual to engage in an activity, to determine that the appropriate person is actually undertaking an activity, or as confirmation that a particular activity has actually be undertaken by an individual or groups of individuals. Still further, the degree to which an individual utilizes the system of the present disclosure could be tied to a score that can be attributed to the individual.

The systems and methods of the present disclosure could also operate in conjuction with non-audio information, such as video or image analysis. For example, the system could monitor one or more videos or photos over time or conduct analysis of a person's facial movements, and such monitoring/analysis could be coupled to the audio analysis features of the present disclosure to further confirm the existence of a pre-defined attribute or condition. Further, monitoring of movements using video or images could be used to assist with analysis of audio analysis (e.g., as confirmation that an attribute detected from an audio sample is accurate). Still further, video/image analysis (e.g., by way of facial recognition or other computer vision techniques) could be utilized as proof of detected voice attributes, or to authenticate that the detected speaker is in fact the actual person speaking.

The various medical conditions capable of being detected by the systems and methods of the present disclosure could be coupled with analysis of the speaker's body position (e.g, supine), which can impact an outcome. Moreover, confirmation of particular positions, or instructions relating to a desired body position of the speaker, could be supplemented using analysis of videos or images by the system.

Advantageously, the detection capabilities of the systems and methods of the present disclosure can detect attributes (e.g., medical conditions or symptoms) that are not evident to individuals, or which are not immediately apparent. For example, the systems and methods can detect minute changes in timbre, frequency spectrum, or other audio characteristics that may not be perceptible to humans, and can use such detected changes (whether immediately detected or detected over time) in order to ascertain whether an attribute exists. Further, even if a single device of the systems of the present disclosure cannot identify a particular voice attribute, a wider network of such devices, each performing voice analysis as discussed herein, may be able to detect such attributes by aggregating information/results. In this regard, the system can create “heat maps” and identify minute disturbances that may merit further attention and resources.

It is further noted that the systems and methods of the present disclosure can be operated to detect and compensate for background noise, in order to obtain better audio samples for analysis. In this regard, the system can cause a device, such as a smart speaker or a smart phone, to emit one or more sounds (e.g., tones, ranges of frequencies, “chirps,” etc.) of pre-defined duration, which can be analyzed by the system to detect acoustic conditions surrounding the speaker and to accommodate for such acoustic conditions, to determine if the speaker is an open or closed environment, to detect whether the environment is noisy or not, etc. The information about the acoustic environment can facilitate applying an appropriate signal enhancement algorithm to a signal degraded by a type of degredation such as noise or reverberation. Other sensor associated with such devices, such as pressure sensors or barometers, can be used to help improve recordings and attendant acoustic conditions. Similarly, the system can sense other environmental conditions that could adversely impact video and image data, and compensate for such conditions. For example, the system could detect, using one or more sensor, whether adverse lighting conditions exist, the direction and intensity of light, whether there is cloud cover, or other environmental conditions, and can adapt a video/image capture device in response so as to mitigate the effects of such adverse conditions. (e.g., by automatically adjusting one or more optical parameters such as white balance, etc.). Such functionality could enhance the ability of the system to detect one or more attributes of a person, such as complexion, age, etc.

The systems and methods of the present disclosure could have wide applicability and usage in conjunction with telemedicine systems. For example, if the system of the present disclosure detect that a person is suffering from a respiratory illness, the system could interface with a telemedicine application that would allow a doctor to remotely examine the person.

Of course, the systems and methods of the present disclosure are not limited to the detection of medical conditions, and indeed, various other attributes such as intoxication, being under the influence of a drug, or a mood could be detected by the system of the present disclosure. In particular, the system could detect whether a person has had too much to drink or is intoxicated (or impaired) by a drug (e.g., cannabis) by analysis of the voice, and alerts and/or actions could be taken by the system in response.

The systems and methods of the present disclosure could prompt an individual to say a particular phrase (e.g., “Hello, world”) at an initial point in time and record such phrase, and at a subsequent point in time, the system could process the recorded phrase using speech-to-text software to convert the recorded phrase to text, then display the text to the user on a display and prompt the user to repeat the text, and then record the phrase again, so that the system obtains two recordings of the person saying precisely the same phrase. Such data could be highly beneficial in allowing the system to detect changes in the person's voice over time. Still further, it is contemplated that the system can couple the audio analysis to a variety of other types of data/analyses, such as phonation and clinical speech results, imagery results (e.g., images of the lungs), notes, diagnoses, or other data.

It is further noted that the systems and methods of the present disclosure can operate with a wide variety of spoken languages. Moreover, the system can be used in conjunction with a wide variety of testing, such as regular medical testing, “drive-by” testing, etc., as well as aerial phenotyping. Additionally, the system need not operate with personally-identifiable information (PII), but is capable of doing so and, in such circumstances, implementing appropriate digital safeguards to protect such PII (e.g., tokenization of sounds to mitigate against data breaches), etc.

The systems and methods of the present disclosure could provide even further benefits. For example, the system could conveniently and rapidly identify intoxication (e.g., by cannabis consumption) and potential impairment related to activities such as driving, tasks occurring during working hours, etc., by analysis of vocal patterns. Moreover, a video camera on a smart phone could be used to capture a video recording along with a detected audio attribute to improve anti-fraud techniques (e.g., to identify the speaker via facial recognition), or to capture movements of the face (e.g., eyes, lips, cheeks, nostrils, etc.) which may be associated with various health conditions. Still further, crowdsourcing of such data might be improved by ensuring users' data privacy (e.g., through the use of encryption, data access control, permission-based controls, blockchain, etc.), offering of incentives (e.g., discounts for items at a pharmacy or grocery-related items), usage of anonymized or categorized data (e.g., scoring or health bands), etc.

Genomic data can be used to match a detected medical condition to a virus strain level to more accurately identify and distinguish geographic paths of a virus based on its mutations over time. Further, vocal pattern data and video data can be used in connection with human resource (HR)-related events, such as to establish a baseline of a healthy person at hiring time, etc. Still further, the system could generate customized alerts for each user relating to permitted geographic locations in response to detected medical conditions (e.g., depending on a detected illness, entry into a theater might not be permitted, but brief grocery shopping might). Additionally, the vocal patterns detected by the system could be linked to health data from previous medical visits, or the health data could be categorized into a score or bands that are then linked to the vocal patterns as metadata. The vocal pattern data could be recorded concurrently with data from a wearable device, which could be used to collect various health condition data such as heart rate, etc.

It is further noted that the systems and methods of the present disclosure could be optimized through the processing of epidemiological data. For example, such data could be utilized to guide processing of particular voice samples from specific populations of individuals, and/or to influence how the voice models of the present disclosure are weighted during processing. Other advantages of using epidemiological information are also possible. Still further, epidemiological could be utilized to control and/or influence the generation and distribution alerts, as well as the dispatching and application of healthcare and other resources as needed.

It is further noted that the system and methods of the present disclosure could process one or more images of an individual's airway or other body part (which could be acquired using a camera of a smart phone and/or using any suitable detection technology, such as optical (visible) light, infrared, ultraviolet, and three-dimensional (3D) data, such as point clouds, light detection and ranging (LiDAR) data, etc.) to detect one or more respiratory or other medical conditions (e.g., using a suitably-trained computer vision technique such as a trained neural network), and one or more actions could be taken in connection with the detected condition(s), such as generating and transmitting an alert to the individual recommending that medical care be obtained to address the condition, tracking the individual's location and/or contacts, or other action.

A significant benefit of the systems and methods of the present disclosure is the ability to gather and analyze voice samples from a multitude of individuals, including individuals who are currently suffering from a respiratory ailment, those who are carrying a pathogen (e.g., a virus) but do not show any symptoms, and those who are not carrying any pathogens. Such a rich collection of data serves to increase the detection capabilities of the systems and methods of the present disclosure (including the voice models thereof).

Still further, it is noted that the systems and methods of the present disclosure can detect medical conditions beyond respiratory ailments through analysis of voice data, such as the onset or current suffering of neurological conditions such as strokes. Additionally, the system can perform archetypal detection of medical conditions (including respiratory conditions) through analysis of coughs, sneezes, and other sounds. Such detection/analysis could be performed using the neural networks described herein, trained to detect neurological and other medical conditions. Still further, the system could be sued to detect and track usage of public transit systems by sick individuals, and/or to control access/usage of such systems by such individuals.

Various incentives could be provided to individuals to encourage such individuals to utilize the systems and methods of the present disclosure. For example, a life insurance company could encourage its insureds to utilize the systems and methods of the present disclosure as part of a self-risk assessment system, and could offer various financial incentives such as reductions in premiums to encourage usage of the system. Governmental bodies could offer tax incentives for individuals who participate in self-monitoring utilizing the systems and methods of the present disclosure. Additionally, businesses could choose to exclude individuals who refuse to utilize the systems/methods of the present disclosure from participating in various business events, activities, benefits, etc. Still further, the systems and methods of the present disclosure could serve as a preliminary screening tool that can be utilized to recommend further, more detailed evaluation by one or more medical professionals.

It is noted that the processes disclosed herein could be triggered by the detection of one or more coughs by an individual. For example, a mobile smartphone could detect the sound of a person coughing, and once detected, could initiate analysis of sounds made by the person (e.g., analysis of vocal sounds, further coughing, etc.) to detect whether the person is suffering from a medical condition. Such detection could be accomplished utilizing an accelerometer or other sensor of the mobile smartphone, or other sensor in communication with the smart phone (e.g., heart rate sensors, etc.), and the detection of coughing by such devices could initiate analysis of sounds made by the person to detect one or more attributes, as disclosed herein. Additionally, time-series degradation capable of being detected by the systems/methods of the present disclosure could provide a rich source of data for conducting community medical surveillance. Even further, the system could discern the number of coughs made by each member of a family in a household, and could utilize such data to identify problematic clusters for further sampling, testing, and analysis. It is also envisioned that the systems and methods of the present disclosure can have significant applicability and usage by healthcare workers at one or more medical facilities (such as hospital nursing staff, doctors, etc.), both to monitor and track exposure of such workers to pathogens (e.g., the new coronavirus causing COVID-19, etc.). Indeed, such workers could serve as a valuable source of reliable data capable of various uses, such as analyzing the transition of workers to infection, analysis of biometric data, and capturing and detecting what ordinary observations and reporting might overlook.

The systems and methods of the present disclosure could be used to perform aggregate monitoring and detection of aggregate degradation of vocal sounds across various populations/networks, whether they be familial, regional, or proximate, in order to determine whether and where to direct further testing resources for the identification of trends and patterns, as well as mitigation (e.g., as part of a surveillance and accreditation system). Even further, the system could provide first responders with advanced notice (e.g., through communication directly to such first responders, or indirectly using some type of service (e.g., 911 service) that communicate with such first responders) of the condition of an individual that is about to be transported to a medical facility, thereby allowing the first responders to don appropriate personal protective equipment (PPE) and/or alter first response practices in the event that the individual is suffering from a highly-communicable illness (such as COVID-19 or other respiratory illness).

It is noted that the functionality described herein could be accessed by way of a web portal that is accessible via a web browser, or by a standalone software application, each executing on a computing device such as a smart phone, personal computer, etc. If a software application is provided, it could also include data collection capabilities, e.g., the ability to capture and store a plurality of voice samples (e.g., taken by recording a person speaking, singing, or coughing into the microphone of a smart phone). Such samples could then be analyzed using the techniques described herein by the software application itself (executing on the smart phone), and/or they could be transmitted to a remote server for analysis thereby. Still further, the systems and methods of the present disclosure could communicate (securely, if desired, using encryption or other secure communication technique) with one or more third-party systems, such as ride-sharing (e.g., UBER) systems so that drivers can determine whether a prospective rider is suffering from a medical condition (or exhibiting attributes associated with a medical condition). Such information could be useful in informing the drivers whether to accept a particular rider (e.g., if the rider is sick), or to take adequate protective measures to protect the drivers before accepting a particular rider. Additionally, the system could detect whether a driver is suffering from a medical condition (or exhibiting attributes associated with a medical condition), and could alert prospective riders of such condition.

Having thus described the system and method in detail, it is to be understood that the foregoing description is not intended to limit the spirit or scope thereof. It will be understood that the embodiments of the present disclosure described herein are merely exemplary and that a person skilled in the art can make any variations and modification without departing from the spirit and scope of the disclosure. All such variations and modifications, including those discussed above, are intended to be included within the scope of the disclosure.

What is claimed is:

1. A machine learning system for detecting at least one voice attribute from input data, comprising:
a processor in communication with a database of input data; and
a predictive voice model executed by the processor, the predictive voice model:
receiving the input data from the database;
processing the input data to identify a speaker of interest from the input data;
isolating one or more predetermined sounds corresponding to the speaker of interest;
generating a plurality of vectors from the one or more predetermined sounds;
generating a plurality of features from the one or more predetermined sounds;
processing the plurality of features to generate a plurality of variables that describe the speaker of interest; and
processing the plurality of variables and vectors to detect the at least one voice attribute.

2. The system of claim 1, wherein the predictive model processes one or more of demographic data, voice data, credit data, lifestyle data, prescription data, social media data, or image data.

3. The system of claim 1, wherein the plurality of vectors comprises a plurality of i-Vectors.

4. The system of claim 3, where the plurality of variables comprises a plurality of functionals that describe the speaker of interest.

5. The system of claim 4, wherein the predictive voice model processes the plurality of iVectors and the plurality of functionals to detect the at least one voice attribute.

6. The system of claim 1, wherein the at least one voice attribute comprises one or more of frequency, perturbation characteristics, tremor characteristics, duration, or timbre.

7. The system of claim 1, wherein the plurality of features comprise mel-frequency cepstral coefficients.

8. The system of claim 1, wherein the at least one voice attribute comprises an indication of whether an individual is a smoker.

9. The system of claim 1, wherein the at least one voice attribute indicates one or more of a respiratory condition, age, gender, general vocal pathology, regional accent, body size, attractiveness, sexuality, social status, personality, emotion, deception, sleepiness, hydration, stress, Sjögren's syndrome, arthritis, dementia, Parkinson's disease, schizophrenia, reflux, alcohol intoxication, epidemiology, cannabis intoxication, blood oxygen levels, a medical condition, a respiratory symptom, a respiratory ailment, an illness, a neurological illness, a neurological disorder, a mood, a physiological characteristic, or an attribute that manifests through perceptible changes in the person's voice.

10. A machine learning method for detecting at least one voice attribute from input data, comprising the steps of:
receiving input data from a database;
processing the input data to identify a speaker of interest from the input data;
isolating one or more predetermined sounds corresponding to the speaker of interest;
generating a plurality of vectors from the one or more predetermined sounds;
generating a plurality of features from the one or more predetermined sounds;
processing the plurality of features to generate a plurality of variables that describe the speaker of interest; and
processing the plurality of variables and vectors to detect the at least one voice attribute.

11. The method of claim 10, further comprising processing one or more of demographic data, voice data, credit data, lifestyle data, prescription data, social media data, or image data.

12. The method of claim 10, wherein the plurality of vectors comprises a plurality of i-Vectors.

13. The method of claim 12, where the plurality of variables comprises a plurality of functionals that describe the speaker of interest.

14. The method of claim 13, further comprising processing the plurality of iVectors and the plurality of functionals to detect the at least one voice attribute.

15. The method of claim 10, wherein the at least one voice attribute comprises one or more of frequency, perturbation characteristics, tremor characteristics, duration, or timbre.

16. The method of claim 10, wherein the plurality of features comprise mel-frequency cepstral coefficients.

17. The method of claim 10, wherein the at least one voice attribute comprises an indication of whether an individual is a smoker.

18. The method of claim 10, wherein the at least one voice attribute indicates one or more of a respiratory condition, age, gender, general vocal pathology, regional accent, body size, attractiveness, sexuality, social status, personality, emotion, deception, sleepiness, hydration, stress, Sjögren's syndrome, arthritis, dementia, Parkinson's disease, schizophrenia, reflux, alcohol intoxication, epidemiology, cannabis intoxication, blood oxygen levels, a medical condition, a respiratory symptom, a respiratory ailment, an illness, a neurological illness, a neurological disorder, a mood, a physiological characteristic, or an attribute that manifests through perceptible changes in the person's voice.

19. A machine learning system for generating one or more vocal metrics from input data, comprising:
a processor receiving at least one voice signal;
a perceptual subsystem executed by the processor, the perceptual subsystem processing the at least one voice signal using a human auditory perception process;
a functionals subsystem executed by the processor, the functionals subsystem processing the at least one voice signal to generate derived functional from the at least one voice signal;
a deep convolutional neural network (CNN) subsystem executed by the processor, the deep CNN subsystem applying one or more CNNs to the at last one voice signal; and
an ensemble model executed by the processor, the ensemble model processing information generated by the perceptual subsystem, the functional subsystem, and the deep CNN subsystem to generate one or more vocal metrics based on the information.

20. The machine learning system of claim 19, wherein the processor performs at least one of digital signal processing, audio segmentation, or speaker diarization on the at least one voice signal.

21. The machine learning system of claim 19, wherein ensemble model processes posterior probabilities generated by the perceptual subsystem, the functional subsystem, and the deep CNN subsystem and associated confidence scores to generate a final prediction.

22. The machine learning system of claim 19, wherein the one or more vocal metrics comprises an indication of whether an individual is a smoker.

23. The machine learning system of claim 19, wherein the one or more vocal metrics indicates one or more of a respiratory condition, age, gender, general vocal pathology, regional accent, body size, attractiveness, sexuality, social status, personality, emotion, deception, sleepiness, hydration, stress, Sjögren's syndrome, arthritis, dementia, Parkinson's disease, schizophrenia, reflux, alcohol intoxication, epidemiology, cannabis intoxication, blood oxygen levels, a medical condition, a respiratory symptom, a respiratory ailment, an illness, a neurological illness, a neurological disorder, a mood, a physiological characteristic, or an attribute that manifests through perceptible changes in the person's voice.

24. A machine learning method for generating one or more vocal metrics from input data, comprising the steps of:
receiving at least one voice signal;
processing the at least one voice signal using a perceptual subsystem executed by a processor, the perceptual subsystem processing the at least one voice signal using a human auditory perception process;
processing the at least one voice signal using a functionals subsystem executed by the processor, the functionals subsystem processing the at least one voice signal to generate derived functional from the at least one voice signal;
processing the at least one voice signal using a deep convolutional neural network (CNN) subsystem executed by the processor, the deep CNN subsystem applying one or more CNNs to the at last one voice signal; and
processing information generated by the perceptual subsystem, the functional subsystem, and the deep CNN subsystem using an ensemble model to generate one or more vocal metrics based on the information.

25. The method of claim 24, further comprising performing at least one of digital signal processing, audio segmentation, or speaker diarization on the at least one voice signal.

26. The method of claim 24, further comprising processing posterior probabilities generated by the perceptual subsystem, the functional subsystem, and the deep CNN subsystem and associated confidence scores to generate a final prediction.

27. The method of claim 24, wherein the one or more vocal metrics comprises an indication of whether an individual is a smoker.

28. The method of claim 24, wherein the one or more vocal metrics indicates one or more of a respiratory condition, age, gender, general vocal pathology, regional accent, body size, attractiveness, sexuality, social status, personality, emotion, deception, sleepiness, hydration, stress, Sjögren's syndrome, arthritis, dementia, Parkinson's disease, schizophrenia, reflux, alcohol intoxication, epidemiology, cannabis intoxication, blood oxygen levels, a medical condition, a respiratory symptom, a respiratory ailment, an illness, a neurological illness, a neurological disorder, a mood, a physiological characteristic, or an attribute that manifests through perceptible changes in the person's voice.
